(19) United States
(12) Patent Application Publication
(10) Pub. No.: US 2026/0269028 A1
(43) Pub. Date: Sep. 10, 2026

(54) System and Method for Multimodal Healthcare Data Compression, Storage, and Harmonization

(71) Applicant: Microsoft Technology Licensing, LLC, Redmond, WA (US)

(72) Inventors: Mustafa Al-Durra, Mississauga (CA); Kumar Thirumalaiah, Karnataka (IN); Ramasubramani Govindan Govindaraj, Chennai (IN); Jason T. Foerch, Hartland, WI (US); Miriam Ney, Bellevue, WA (US); Bhuvana Sudha Bellala, Shoreline, WA (US); Devendra Kumar, Snoqualmie, WA (US); Matthew Anthony Dearing, Fishers, IN (US); Subramanian Janardanan, Redmond, WA (US); Umesh Rustogi, Bellevue, WA (US); Venkiteswaran Balakrishnan, San Jose, CA (US); Madhuchhanda Bhowal, Kirkland, WA (US)

(21) Appl. No.: 19/075,122

(22) Filed: Mar. 10, 2025

(51) Int. Cl. G16H 10/60; G06F 16/21; G06F 16/22; G06F 16/25; G16H 30/20

(52) U.S. Cl. CPC G16H 10/60; G06F 16/212; G06F 16/2282; G06F 16/258; G16H 30/20

(57) ABSTRACT

A method, computer program product, and computing system for compressing first modality data in a first processing datastore by: compressing the first modality data into a first delta table of a first schema, and storing the first delta table of the first schema in the first processing datastore. The first modality data is transformed into normalized first modality data by: transforming the first delta table of the first schema into a first delta table of a second schema, and storing the second delta table of the second schema in a second processing datastore. The second modality data is compressed in the first processing datastore. The second modality data is transformed into normalized second modality data. The normalized first modality data is associated with the normalized second modality data in the second processing datastore.

### BACKGROUND

Cloud computing systems process immense amounts of data each day, with estimates suggesting that the amount can reach 1 billion gigabytes. For example, healthcare systems handle enormous amounts of data daily, from different sources, in multiple formats and modalities which amount to about 30% of all the world's data. Managing the storage and access of multimodal data (i.e., data from multiple modalities and source systems) is challenging in many data storage solutions because of the unique properties of each data modality. For instance, when breaking down data silos in the healthcare space, organizations can make better decisions and advance their research to pave the way for new medical discoveries and precision medicine. Additionally, having access to harmonized image and clinical data about a patient helps organizations improve their treatments with more customized solutions that account for multimodal patterns. Further, unified access to multimodal data allows organizations to deploy artificial intelligence (AI) models to predict prognosis and diagnoses of disease.

In the context of the healthcare space, the challenge in creating a harmonized data estate stems from the complexity of healthcare data. The volume of data required for robust AI analytics is immense, computationally heavy, and managing this data requires sophisticated data governance, security, and privacy.

### BRIEF DESCRIPTION OF THE DRAWINGS

FIG. 1 is a flow chart of one implementation of a multimodal data management process;

FIG. 2 is a diagrammatic view of the multimodal data management process of FIG. 1; and

FIG. 3 is a diagrammatic view of a cloud system and the multimodal data management process coupled to a distributed computing network.

Like reference symbols in the various drawings indicate like elements.

### DETAILED DESCRIPTION OF THE EMBODIMENTS

Implementations of the present disclosure enable a cloud-scale, multimodal data platform that manages the rapid growth of heterogeneous medical data (e.g., clinical and image data) and is flexible enough to incorporate new data types and standards as they become available. For example, the multimodal data management process compresses multimodal data (e.g., first modality data and second modality data) in their respective formats in a first datastore of a data lake. Certain data modalities, like image data or video data, can require significant amounts of storage space. Aggregating different data modalities in a single datastore using conventional approaches requires siloed datastores for different data modalities and combining subsequently. These approaches are vulnerable to multimodal data integrity and compatibility issues as separate data modalities may be subject to different types of compression or truncation that result in poor connections between data of one modality and data of another modality (e.g., a patient's electronic health record (EHR) and medical imaging systems). Accordingly, the present disclosure provides a process for systematic, multi-layered processing of multimodal data that compresses each data modality while enhancing the structure and combinability of the multimodal data.

Each modality data is compressed into delta tables of a first predefined schema. The compressed modality data is transformed into normalized data in the form of delta tables of a second predefined schema. This normalized modality data is stored in a second datastore of the data lake. The normalized modality data is converted to enhanced modality data by converting the delta tables of the second predefined schema to delta tables of a third predefined schema. The enhanced modality data is stored in a third datastore of the data lake. The normalized modality data in the second predefined schema and the enhanced modality data in the third predefined schema allows multimodal data to be associated with data of other modalities and processed using a multimodal data operation. For example, a multimodal query can be performed on the normalized modality data and/or the aggregated enhanced modality data. In another example, a machine learning model can process multimodal data from the combination of the normalized modality data and/or the enhanced modality data.

The details of one or more implementations are set forth in the accompanying drawings and the description below. Other features and advantages will become apparent from the description, the drawings, and the claims.

### The Multimodal Data Management Process

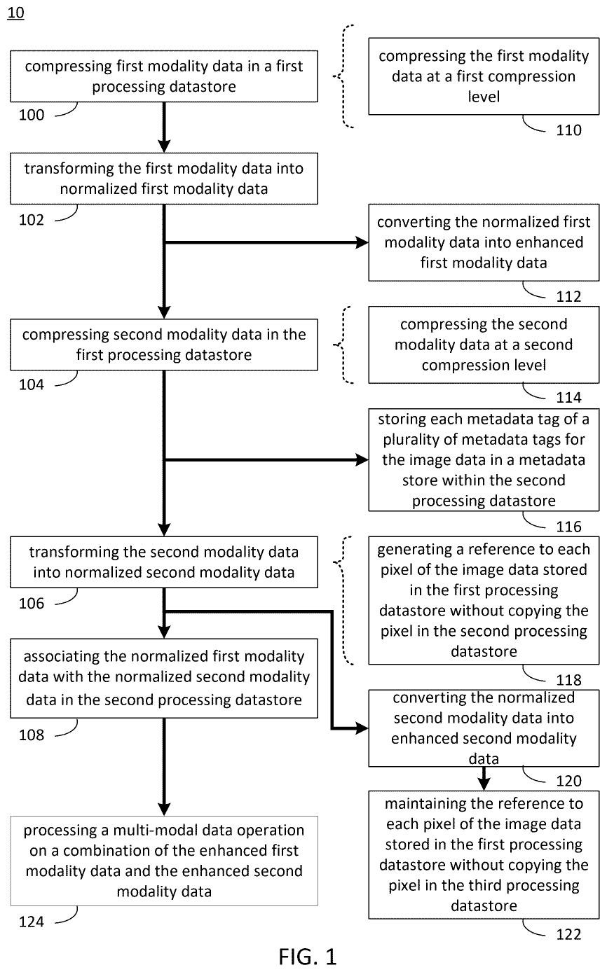

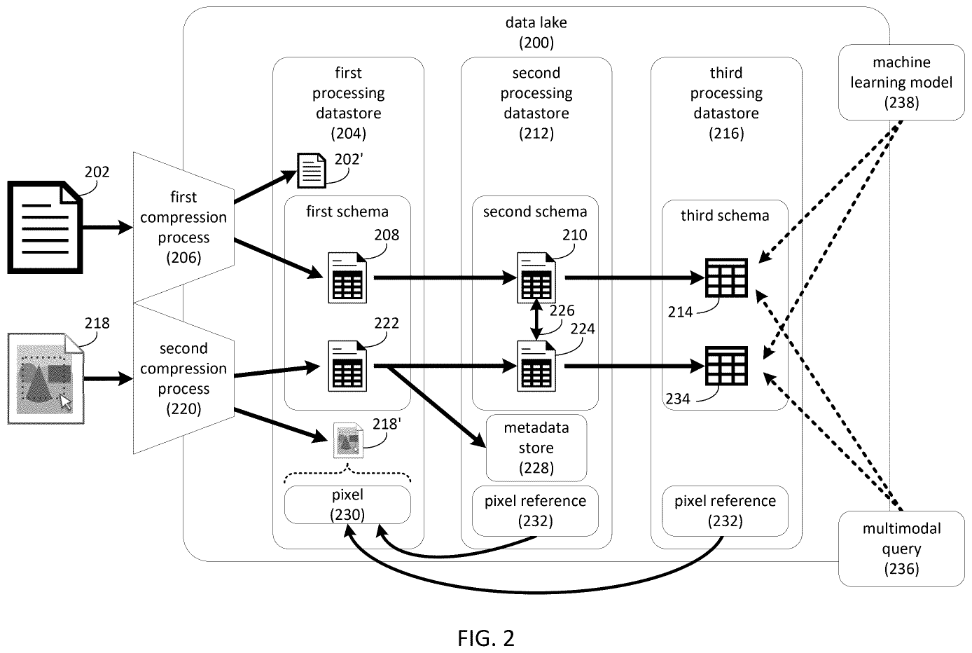

Referring to FIGS. 1-2, multimodal data management process 10 compresses 100 first modality data in a first processing datastore by: compressing the first modality data into a first delta table of a first schema and storing the first delta table of the first schema in the first processing datastore. The first modality data is transformed 102 into normalized first modality data by: transforming the first delta table of the first schema into a first delta table of a second schema and storing the second delta table of the second schema in a second processing datastore. The second modality data is compressed 104 in the first processing datastore by: extracting a metadata tag from the second modality data into a second delta table of the first schema and storing the second delta table of the first schema in the first processing datastore. The second modality data is transformed 106 into normalized second modality data by: transforming the second delta table of the first schema into a second delta table of the second schema and storing the second delta table of the second schema in the second processing datastore. The normalized first modality data is associated 108 with the normalized second modality data in the second processing datastore.

In some implementations, multimodal data management process 10 compresses 100 first modality data in a first processing datastore. Modality data is data of a particular form or type. Examples of modality data include text data, image data, audio data, video data. In some implementations, modality data is defined as data having a distinct type for formatting. In some implementations, modality data includes a set of data used in a particular field (e.g., clinical data, sensor data, financial data, legal data, etc.). In one example, first modality data (i.e., one of a set of multiple modalities of data) is clinical data. Clinical data includes medical information (e.g., patient demographics, medical history information, laboratory test results, clinical trial data, medication information, etc.) associated with an individual or group of individuals. As will be discussed in greater detail below, upon ingestion into a data lake, first modality data may include various formats of clinical data (e.g., different types of text files or formats for electronic health records (EHRs)). Accordingly, multimodal data management process 10 processes first modality data for storage in a multi-layered processing datastore.

In some implementations, compressing 100 the first modality data includes compressing 110 the first modality data at a first compression level. For example, compressing the first modality data includes encoding the first modality data from its initial state and size into a compressed data type or format associated with the first modality. In one example, first modality data is clinical data and includes an electronic health record associated with a particular person. In this example, multimodal data management process 10 compresses 100 the first modality data using a data compression algorithm. Examples of data compression algorithms include Huffman coding, Run-Length Encoding (RLE), Discrete Cosine Transform (DCT).

In some implementations, multimodal data management process 10 compresses 100 the first modality data by compressing the first modality data into a first delta table of a first schema. In one example, multimodal data management process 10 compresses the first modality data into a delta table. A delta table is a data structure used in data lakes and is designed to support Atomicity, Consistency, Isolation, and Durability (ACID) transactions, ensuring data integrity and reliability during concurrent operations. Additionally, delta tables enforce schema constraints (e.g., types of data for each entry of the schema). In one example, the delta table is a Parquet file. A Parquet file is a columnar storage file format optimized for use with data processing frameworks and is designed to efficiently store and process large datasets, providing both performance and storage benefits.

In some implementations, a schema is an organization of data within tables of the first schema, including the data types, constraints, and relationships between different data elements. For example, the schema defines the columns in each table and their respective data types (e.g., integer, string, date). This ensures that data is stored in each delta table in a consistent format. In one example, the first schema is a staging table schema. For example, staging tables in the first datastore include a predefined number of columns and are designed to hold each data modality and format in a single table. In another example, the first schema is the Parquet table schema that defines the structure (i.e., names, types and value types for fields) within Parquet files. As will be discussed in greater detail below, the staged processing of multimodal data management process 10 allows multimodal data to be compressed and normalized in steps across different schemas. A common schema shared by both data modalities enable multimodal data operations to be performed on the combination of first modality data and second modality data.

In some implementations, the compression of first modality data is defined by a data compression level. The compression level is defined by the type or modality of the data being compressed. In one example with clinical data as the first modality data, multimodal data management process 10 compresses 110 the first modality data at a compression level of up to 75%. However, it will be appreciated that other compression levels may be used and are dependent upon the compression algorithm and the modality of the data. In some implementations, the compression level is configurable by a user (e.g., using a user interface and/or by selecting a particular compression level or compression algorithm).

In some implementations, multimodal data management process 10 compresses the first modality data into a first delta table of a first schema and stores the first delta table of the first schema in a first processing datastore. Referring also to FIG. 2 and in some implementations, a data lake is a centralized repository designed to store, process, and secure large volumes of structured, semi-structured, and unstructured data. Data lake 200 allows data to be stored in various forms until it is processed for analysis, providing flexibility and scalability. In some implementations, multimodal data management process 10 processes first modality data (e.g., first modality data 202) for storage in a first processing datastore (e.g., first processing datastore 204) within data lake 200 using a first compression process (e.g., first compression process 206) to generate compressed first modality data 202′ and a delta table of a first predefined schema (e.g., delta table 208).

In one example, first processing datastore 204 is a first lakehouse of data lake 200. In some implementations, a lakehouse is a subset of a data lake. In some implementations, first processing datastore 204 stores first modality data in its raw form (following compression by first compression process 206). In this example and as will be discussed in greater detail below, each processing datastore stores modality data in different stages of processing (e.g., raw, normalized, and enhanced).

In some implementations, multimodal data management process 10 transforms 102 the first modality data into normalized first modality data by transforming the first delta table of the first schema into a first delta table of a second schema and storing the first delta table of the second schema in a second processing datastore. For example, first modality data 202 stored in first processing datastore includes a compressed version of the initial first modality data and a delta table in the first schema (e.g., delta table 208). Multimodal data management process 10 transforms 102 first modality data 202 from first processing datastore 204 to normalized first modality data (e.g., normalized first modality data 210). In some implementations, normalized first modality data 210 is a representation of first modality data 202 that adheres to a second predefined schema. For example, first modality data stored in first processing datastore 204 may be defined in a first schema that lacks the mapping to other data modalities. Accordingly, multimodal data management process 10 transforms 102 first modality data 202 into normalized first modality data by transforming first modality data into delta table(s) of a second, predefined schema.

In one example, the second schema concerns clinical data with a key-value pair, where each key describes what a corresponding value represents (e.g., PatientID(integer), VisitDate(date), Diagnosis(string), Treatment(string), DoctorID(integer)). In another example, the second schema concerns e-commerce data with a key-value pair, where each key describes what a corresponding value represents (e.g., OrderID(integer), CustomerID(integer), ProductID(integer), OrderDate(date), Quantity(integer), TotalPrice(decimal)). In this example, the second schema may be the Fast Healthcare Interoperability Resources® (FHIR) schema. The FHIR schema is a standard developed by Health Level Seven International (HL7) for the electronic exchange of healthcare information and is designed to facilitate interoperability between healthcare systems. The FHIR schema provides a common data language that enables data analysts, data scientists, and developers to collaborate and build data-driven solutions that improve patient outcomes and business performance. This schema supports data from across different healthcare business domains such as clinical, administrative, financial, and social. Accordingly, the transformation of modality data to delta tables of the FHIR schema captures data defined by the FHIR standard by making FHIR resources available as tables and columns within the second processing datastore. In one example, by flattening the FHIR information from first modality data 202 into delta tables, multimodal data management process 10 enables the use of tools like T-SQL® and Spark SQL® to explore and analyze the multimodal data.

In some implementations, multimodal data management process 10 transforms 102 the first modality data into normalized first modality data by storing the first delta table of the second schema in a second processing datastore. As discussed above and in some implementations, the second processing datastore is a second lakehouse of the data lake (e.g., data lake 200). In one example, second processing datastore 212 stores first modality data (e.g., first delta table of the second schema) in a normalized form relative to the first schema. In one example, multimodal data management process 10 processes a predefined mapping of first modality data to corresponding delta table entries of the first schema to transform 102 first modality data 202 to normalized first modality data 210. In another example, multimodal data management process 10 processes first modality data 202 using a trained machine learning model to map delta table entries in delta tables of the first schema from first modality data 202 to corresponding entries in delta table(s) of the second schema for normalized first modality data 210. In another example, multimodal data management process 10 processes first modality data 202 using a configurable template to map delta table entries in delta tables of the first schema from first modality data 202 to corresponding entries in delta table(s) of the second schema for normalized first modality data 210.

In some implementations, multimodal data management process 10 converts 112 the normalized first modality data into enhanced first modality data by converting the first delta table of the second schema into a first delta table of a third schema and storing the first delta table of the third schema in a third processing datastore. For example, normalized first modality data 210 stored in second processing datastore is a normalized version of the compressed first modality data. However, normalized first modality data 210 may be defined in a second schema that is unable to aggregate data from other data modalities. Accordingly, multimodal data management process 10 converts 112 normalized first modality data 210 from second processing datastore 212 to enhanced first modality data (e.g., enhanced first modality data 214). In some implementations, enhanced first modality data 214 is a representation of normalized first modality data 210 that adheres to a third schema by mapping key-value pairs across data modalities. For example, multimodal data management process 10 converts 112 normalized first modality data 210 into enhanced first modality data by converting the delta table(s) of the second schema to a delta table of the third schema.

In some implementations, the third schema is Observational Medical Outcomes Partnership (OMOP). OMOP is a schema designed for standardizing observational health data for research and analytics. The OMOP schema provides a common data model (CDM) that allows users to conduct large-scale observational studies and comparative effectiveness research. As discussed above and in one example, normalized first modality data defines a delta table(s) of the second schema (e.g., FHIR). In this example, the FHIR schema is resource-based, with each resource representing a specific healthcare concept (e.g., patient, observation, medication). By contrast, OMOP is a comprehensive data model that is structured to support complex queries and analyses, making it suitable for research purposes. Accordingly, enhanced modality data (e.g., enhanced first modality data 214) includes modality data that is converted from a normalized data to delta tables of a third schema that also map to other modalities. In one example, multimodal data management process 10 processes a predefined mapping of delta table entries of the second schema to corresponding delta table entries of the third schema to convert 112 normalized first modality data 210 to enhanced first modality data 214. In another example, multimodal data management process 10 processes normalized first modality data 210 using a trained machine learning model to map delta table entries in delta tables of the second schema from normalized first modality data 210 to corresponding entries in delta table(s) of the third schema for enhanced first modality data 214. In another example, multimodal data management process 10 processes normalized first modality data 210 using a configurable template to map delta table entries in delta tables of the second schema from normalized first modality data 210 to corresponding entries in delta table(s) of the third schema for enhanced first modality data 214.

In some implementations, multimodal data management process 10 converts 112 the normalized first modality data into enhanced first modality data by storing the first delta table of the third schema in a third processing datastore. As discussed above and in some implementations, the third processing datastore (e.g., third processing data store 214) is a third lakehouse of the data lake (e.g., data lake 200). In one example, third processing datastore 216 stores enhanced first modality data in an enhanced form relative to the second schema (e.g., first delta table of the third schema). As discussed above and in some implementations, the third schema allows multimodal data (e.g., first multimodal data and second multimodal data) to be aggregated and processed in a multimodal data operation.

In some implementations, multimodal data management process 10 compresses 104 second modality data in the first processing datastore. For example, in addition to a first modality data, multimodal data management process 10 processes second modality data (e.g., second modality data 218) that is data of a different modality than first modality data 202. In one example, the second modality data is image data. Compared to clinical data, for example, image data is typically stored in different file formats than clinical data. Additionally, image data generally consumes more storage space within a datastore than clinical data. In some implementations, image data is generated and stored separately than clinical data. For example, medical images produced by medical imaging machines (e.g., an X-ray machine or a magnetic resonance imaging (MRI) machine) are typically managed by the respective machine vendors or by the software operating the particular imaging machine. Accordingly, associating first modality data (e.g., clinical data) to related second modality data (e.g., image data) may not be possible from a comparison of each modality data separately in its raw format.

In some implementations, compressing 104 the second modality data includes compressing 114 the second modality data at a second compression level. As discussed above, the compression level for the second modality data is defined by the type or modality of the data being compressed. Continuing with the example of second modality data being image data, multimodal data management process 10 compresses 114 second modality data 218 at a second compression level for image data. In one example, multimodal data management process 10 compresses 116 image data by compressing all related image data in a folder or group-based compression. In another example, multimodal data management process 10 compresses 116 image data in a lossless manner as PNG or GIF format. In another example, multimodal data management process 10 compresses 114 image data in a lossy manner as JPEG format. However, it will be appreciated that other compression levels may be used when compressing second modality data. In some implementations, multimodal data management process 10 compresses 106 second modality data into a Parquet file. Referring again to FIG. 2, multimodal data management process 10 processes second modality data (e.g., second modality data 202) for storage in a first processing datastore (e.g., first processing datastore 204) within data lake 200 using a second compression process (e.g., second compression process 220) to store compressed second modality data 218′ and delta tables of the first schema (e.g., delta table 222).

In some implementations, multimodal data management process 10 compresses 104 the second modality data in the first processing datastore by extracting a metadata tag from the second modality data into a second delta table of the first schema and storing the second delta table of the first schema in the second processing datastore. Continuing with the above example, second modality data includes image data within the healthcare space. In this example and in some implementations, the image data is generated in an initial data format (e.g., Digital Imaging and Communications in Medicine (DICOM)). DICOM is a standard for transmitting, storing, and sharing medical images and related information. The DICOM standard facilitates the integration of medical imaging devices, such as scanners, servers, workstations, and network hardware, from multiple manufacturers. In some implementations, the DICOM standard or schema defines a plurality of tags (e.g., over 5,000 tags) that include standardized tags (e.g., “patientID”, “numberOfStudyRelatedSeries”, “instanceNumber”, etc.) and vendor-specific tags. In this example, multimodal data management process 10 compresses 104 the DICOM data of second modality data into delta tables of the first schema (e.g., staging tables or Parquet files) and stores the delta table (e.g., delta table 222) in first processing datastore 204.

In some implementations, multimodal data management process 10 transforms 106 the second modality data into normalized second modality data by transforming the second delta table of the first schema into a second delta table of the second schema and storing the second delta table of the second schema in the second processing datastore. For example, multimodal data management process 10 transforms 106 second modality data 218 into normalized second modality data (e.g., normalized second modality data 224) by processing a predefined mapping of delta table entries of the first schema to corresponding delta table entries of the second schema to transform 106 second modality data 218 to normalized second modality data 224. In another example, multimodal data management process 10 processes second modality data 218 using a trained machine learning model to map metadata tags to corresponding entries in delta table(s) of the first schema for normalized second modality data 224. In another example, multimodal data management process 10 processes second modality data 218 using an extensible configuration to map metadata tags to corresponding entries in delta table(s) of the first schema for normalized second modality data 224.

In some implementations, multimodal data management process 10 associates 108 the normalized first modality data with the normalized second modality data in the second processing datastore. Associating 108 normalized first modality data 210 with normalized second modality data 224 includes linking particular portions of normalized first modality data 210 with corresponding portions of normalized second modality data 224. For example, suppose normalized first modality data 210 includes EHR data for a particular person and normalized second modality data 224 includes medical imaging dataset referenced by the EHR data. In this example as normalized first modality data 210 and normalized second modality data 224 are within second processing datastore 212 and are “normalized” to be in the same schema, multimodal data management process 10 associates the relevant medical imaging dataset of normalized second modality data 224 with the EHR data of normalized first modality data 210. In some implementations, associating 108 normalized first modality data 210 and normalized second modality data 224 includes generating an entry in a table within second processing datastore 212 indicating the association of normalized first modality data 210 and normalized second modality data 224. Referring again to FIG. 2, an association between normalized first modality data 210 and normalized second modality data 224 is represented using the line between normalized first modality data 210 and normalized second modality data 224 (e.g., association 226).

In some implementations, multimodal data management process 10 stores 116 each metadata tag of a plurality of metadata tags for the image data in a metadata store within the second processing datastore. For example, when processing image data as the second modality data, multimodal data management process 10 retains every metadata tag of the second modality data in a metadata store (e.g., metadata store 228). In some implementations, metadata store 228 is a data structure within the second processing datastore (e.g., second processing datastore 212). In this manner, multimodal data management process 10 maintains each and every metadata tag from the second modality data as it is processed (e.g., normalized and enhanced).

In some implementations, transforming the second modality data into the normalized second modality data includes generating 118 a reference to each pixel of the image data stored in the first processing datastore without copying the pixel in the second processing datastore. For example, when transforming second modality data 218 into normalized second modality data, instead of copying each pixel of the image data stored in first processing datastore 204, multimodal data management process 10 generates 118 a reference (e.g., pixel reference 230) to the pixel (e.g., pixel 232) stored in first processing datastore 204. In this manner, as second modality data 218 is normalized and enhanced, pixels are not copied through each processing datastore but a reference to each pixel (e.g., pixel reference 230) in first processing datastore 204 is propagated through data lake 200. Accordingly, multimodal data management process 10 reduces the amount of storage space required by image data of second modality data using references back to the pixel stored in first processing datastore 204.

In some implementations, multimodal data management process 10 converts 120 the normalized second modality data into enhanced second modality data by converting the second delta table of the first schema into a second delta table of the second schema and storing the second delta table of the second schema in the third processing datastore. As discussed above, normalized second modality data 224 stored in second processing datastore is a normalized version of the compressed second modality data. However, normalized second modality data 224 may be defined in the first schema that lacks the ability to be aggregated with data of other data modalities (e.g., first modality data 202). Accordingly, multimodal data management process 10 converts 120 normalized second modality data 224 from second processing datastore 212 to enhanced second modality data (e.g., enhanced second modality data 234). In some implementations, enhanced second modality data 234 is a representation of second modality data 224 that adheres to the third schema by mapping key-value pairs across data modalities. For example, multimodal data management process 10 converts 120 normalized second modality data 224 into enhanced second modality data by converting the delta table(s) of the second schema to a delta table of the third schema.

As discussed above and in some implementations, the third schema is OMOP which allows second modality data to be enhanced in OMOP's comprehensive data model structured to support complex queries and analyses. Accordingly, enhanced second modality data (e.g., enhanced second modality data 234) includes second modality data that is converted from a normalized data to delta table(s) of a third schema that also map to other modalities. In one example, multimodal data management process 10 processes a predefined mapping of delta table entries of the second schema to corresponding delta table entries of the third schema to convert 120 normalized second modality data 224 to enhanced second modality data 234. In another example, multimodal data management process 10 processes normalized second modality data 224 using a trained machine learning model to map delta table entries in delta tables of the second schema from normalized second modality data 224 to corresponding entries in delta table(s) of the third schema for enhanced second modality data 234. In another example, multimodal data management process 10 processes normalized second modality data 224 using an extensible configuration to map delta table entries in delta tables of the second schema from normalized second modality data 224 to corresponding entries in delta table(s) of the third schema for enhanced second modality data 234.

In some implementations, converting 110 the normalized second modality data into enhanced second modality data includes maintaining 122 the reference to each pixel of the image data stored in the first processing datastore without copying the pixel in the third processing datastore. For example and as discussed above, multimodal data management process 10 generates pixel reference 230 to avoid copying pixel 232 in each of second processing datastore 212 and third processing datastore 216. Accordingly, multimodal data management process 10 maintains 122 pixel reference 230 in third processing datastore 216 to pixel 232 stored in first processing datastore 204 during the conversion of normalized second modality data 224 into enhanced second modality data 234.

In some implementations, multimodal data management process 10 processes 124 a multimodal data operation on a combination of the enhanced first modality data and the enhanced second modality data using the association of the enhanced first modality data and the enhanced second modality data in the third processing datastore. For example, with enhanced first modality data 214 and enhanced second modality data 234 and the association therebetween, multimodal data management process 10 processes 124 a multimodal data operation on the combination of enhanced first modality data 214 and enhanced second modality data 234. In one example, multimodal data management process 10 processes 124 a multimodal query (e.g., multimodal query 236) from a user or computing device. Multimodal query 228 is a query executed on third processing datastore 216 that retrieves enhanced first modality data 214 and/or enhanced second modality data 234. In some implementations, processing 124 the multimodal data operation includes processing enhanced first modality data 214 and enhanced second modality data 234 using a machine learning model (e.g., machine learning model 238). In this example, multimodal data management process 10 provides the association between enhanced first modality data 214 and enhanced second modality data 234 to establish connections between inputs of machine learning model 238. Accordingly, multimodal data management process 10 allows multimodal data storage and access by processing multimodal data through multiple stages of processing to compress, normalize, and enhance the multimodal data.

### System Overview

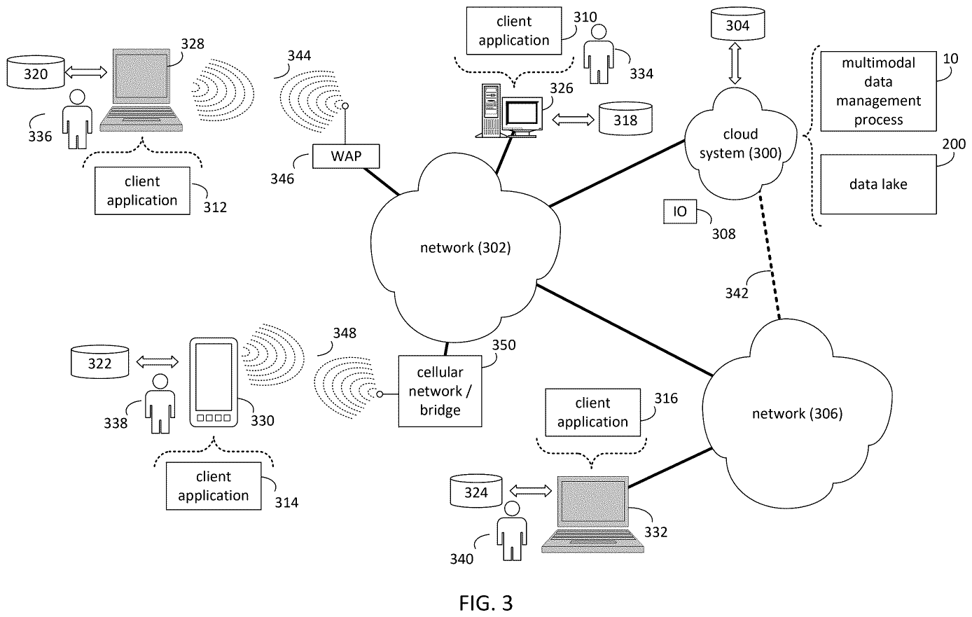

Referring to FIG. 3, a multimodal data management process 10 is shown to reside on and is executed by cloud system 300, which is connected to network 302 (e.g., the Internet or a local area network). Examples of cloud system 300 include: a Network Attached Storage (NAS) system, a Storage Area Network (SAN), a personal computer with a memory system, a server computer with a memory system, and a cloud-based device with a memory system. A SAN includes one or more of a personal computer, a server computer, a series of server computers, a minicomputer, a mainframe computer, a RAID device, and a NAS system.

The various components of cloud system 300 execute one or more operating systems, examples of which include: Microsoft® Windows®; Mac® OS X®; Red Hat® Linux®, Windows® Mobile, Chrome OS, Blackberry OS, Fire OS, or a custom operating system (Microsoft and Windows are registered trademarks of Microsoft Corporation in the United States, other countries or both; Mac and OS X are registered trademarks of Apple Inc. in the United States, other countries or both; Red Hat is a registered trademark of Red Hat Corporation in the United States, other countries or both; and Linux is a registered trademark of Linus Torvalds in the United States, other countries or both).

The instruction sets and subroutines of multimodal data management process 10, which are stored on storage device 304 included within cloud system 300, are executed by one or more processors (not shown) and one or more memory architectures (not shown) included within cloud system 300. Storage device 304 may include: a hard disk drive; an optical drive; a RAID device; a random-access memory (RAM); a read-only memory (ROM); and all forms of flash memory storage devices. Additionally or alternatively, some portions of the instruction sets and subroutines of multimodal data management process 10 are stored on storage devices (and/or executed by processors and memory architectures) that are external to cloud system 300.

In some implementations, network 302 is connected to one or more secondary networks (e.g., network 306), examples of which include: a local area network; a wide area network; or an intranet.

Various input/output (IO) requests (e.g., IO request 308) are sent from client applications 310, 312, 314, 316 to cloud system 300. Examples of IO request 308 include data write requests (e.g., a request that content be written to cloud system 300) and data read requests (e.g., a request that content be read from cloud system 300).

The instruction sets and subroutines of client applications 310, 312, 314, 316, which may be stored on storage devices 318, 320, 322, 324 (respectively) coupled to client electronic devices 326, 328, 330, 332 (respectively), may be executed by one or more processors (not shown) and one or more memory architectures (not shown) incorporated into client electronic devices 326, 328, 330, 332 (respectively). Storage devices 318, 320, 322, 324 may include: hard disk drives; tape drives; optical drives; RAID devices; random access memories (RAM); read-only memories (ROM), and all forms of flash memory storage devices. Examples of client electronic devices 326, 328, 330, 332 include personal computer 326, laptop computer 328, smartphone 330, laptop computer 332, a server (not shown), a data-enabled, and a dedicated network device (not shown). Client electronic devices 326, 328, 330, 332 each execute an operating system.

Users 334, 336, 338, 340 may access cloud system 300 directly through network 302 or through secondary network 306. Further, cloud system 300 may be connected to network 302 through secondary network 306, as illustrated with link line 342.

The various client electronic devices may be directly or indirectly coupled to network 302 (or network 306). For example, personal computer 326 is shown directly coupled to network 302 via a hardwired network connection. Further, laptop computer 332 is shown directly coupled to network 306 via a hardwired network connection. Laptop computer 328 is shown wirelessly coupled to network 302 via wireless communication channel 344 established between laptop computer 328 and wireless access point (e.g., WAP) 346, which is shown directly coupled to network 302. WAP 346 may be, for example, an IEEE 802.11a, 802.11b, 802.11g, 802.11n, Wi-Fi®, and/or Bluetooth® device that is capable of establishing a wireless communication channel 344 between laptop computer 328 and WAP 346. Smartphone 330 is shown wirelessly coupled to network 302 via wireless communication channel 348 established between smartphone 330 and cellular network/bridge 350, which is shown directly coupled to network 302.

### General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be used. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object-oriented programming language. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet.

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general-purpose computer/special purpose computer/other programmable data processing apparatus, such that the instructions, which execute via the processor of the computer or other programmable data processing apparatus, create means for implementing the functions/acts specified in the flowchart and/or block diagram block or blocks.

These computer program instructions may also be stored in a computer-readable memory that may direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, not at all, or in any combination with any other flowcharts depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
compressing first modality data in a first processing datastore by:
compressing the first modality data into a first delta table of a first schema, and
storing the first delta table of the first schema in the first processing datastore;
transforming the first modality data into normalized first modality data by:
transforming the first delta table of the first schema into a first delta table of a second schema, and
storing the first delta table of the second schema in a second processing datastore;
compressing second modality data in the first processing datastore by:
extracting a metadata tag from the second modality data into a second delta table of the first schema, and
storing the second delta table of the first schema in the first processing datastore;
transforming the second modality data into normalized second modality data by:
transforming the second delta table of the first schema into a second delta table of the second schema, and
storing the second delta table of the second schema in the second processing datastore; and
associating the normalized first modality data with the normalized second modality data in the second processing datastore.

2. The computer-implemented method of claim 1, further comprising:
converting the normalized first modality data into enhanced first modality data by:
converting the first delta table of the second schema into a first delta table of a third schema, and
storing the first delta table of the third schema in a third processing datastore.

3. The computer-implemented method of claim 2, further comprising:
converting the normalized second modality data into enhanced second modality data by:
converting the second delta table of the second schema into a second delta table of the third schema, and
storing the second delta table of the third schema in the third processing datastore.

4. The computer-implemented method of claim 3, further comprising:
processing a multimodal data operation on a combination of the enhanced first modality data and the enhanced second modality data.

5. The computer-implemented method of claim 1, wherein the first processing datastore is a first lakehouse of a data lake, the second processing datastore is a second lakehouse of the data lake, and the third processing datastore is a third lakehouse of the data lake.

6. The computer-implemented method of claim 1, wherein the first modality data is clinical data.

7. The computer-implemented method of claim 6, wherein the second modality data is image data.

8. The computer-implemented method of claim 7, wherein the second schema is Fast Healthcare Interoperability Resources® (FHIR).

9. The computer-implemented method of claim 8, wherein the third schema is Observational Medical Outcomes Partnership (OMOP).

10. The computer-implemented method of claim 7, further comprising:
storing each metadata tag of a plurality of metadata tags for the image data in a metadata store within the second processing datastore.

11. The computer-implemented method of claim 7, wherein transforming the second modality data into the normalized second modality data includes generating a reference to each pixel of the image data stored in the first processing datastore without copying the pixel in the second processing datastore.

12. The computer-implemented method of claim 11, wherein converting the normalized second modality data into enhanced second modality data includes maintaining the reference to each pixel of the image data stored in the first processing datastore without copying the pixel in the third processing datastore.

13. The computer-implemented method of claim 7, wherein the image data is Digital Imaging and Communications in Medicine (DICOM) data.

14. The computer-implemented method of claim 1, wherein compressing the first modality data includes compressing the first modality data at a first compression level and wherein compressing the second modality data includes compressing the second modality data at a second compression level.

15. A computing system comprising:
a memory; and
a processor configured to:
compress first modality data in a first processing datastore by:
compressing the first modality data into a first delta table of a first schema, and
storing the first delta table of the first schema in the first processing datastore, wherein the first modality data is clinical data;
transform the first modality data into normalized first modality data by:
transforming the first delta table of the first schema into a first delta table of a second schema, and
storing the first delta table of the second schema in a second processing datastore;
compress second modality data in the first processing datastore by:
extracting a metadata tag from the second modality data into a second delta table of the first schema, and
storing the second delta table of the first schema in the first processing datastore, wherein the second modality data is image data;
transform the second modality data into normalized second modality data by:
transforming the second delta table of the first schema into a second delta table of the second schema, and
storing the second delta table of the second schema in the second processing datastore; and
associate the normalized first modality data with the normalized second modality data in the third processing datastore.

16. The computing system of claim 15, wherein the processor is further configured to:
convert the normalized first modality data into enhanced first modality data by:
converting the first delta table of the second schema into a first delta table of a third schema, and
storing the first delta table of the third schema in a third processing datastore.

17. The computing system of claim 16, wherein the processor is further configured to:
convert the normalized second modality data into enhanced second modality data by:
converting the second delta table of the second schema into a second delta table of the third schema, and
storing the second delta table of the third schema in the third processing datastore.

18. The computing system of claim 17, wherein the processor is further configured to:
process a multimodal data operation on a combination of the enhanced first modality data and the enhanced second modality data.

19. The computing system of claim 17, wherein the second schema is Fast Healthcare Interoperability Resources® (FHIR), and the third schema is Observational Medical Outcomes Partnership (OMOP).

20. A computer program product residing on a computer readable medium having a plurality of instructions stored thereon which, when executed by a processor, cause the processor to perform operations comprising:
compressing first modality data in a first processing datastore by:
compressing the first modality data into a first delta table of a first schema, and
storing the first delta table of the first schema in the first processing datastore;
transforming the first modality data into normalized first modality data by:
transforming the first delta table of the first schema into a first delta table of a second schema, and
storing the first delta table of the second schema in a second processing datastore;
converting the normalized first modality data into enhanced first modality data by:
converting the first delta table of the second schema into a first delta table of a third schema, and
storing the first delta table of the third schema in a third processing datastore;
compressing second modality data in the first processing datastore by:
extracting a metadata tag from the second modality data into a second delta table of the first schema, and
storing the second delta table of the first schema in the first processing datastore;
transforming the second modality data into normalized second modality data by:
transforming the second delta table of the first schema into a second delta table of the second schema, and
storing the second delta table of the second schema in the second processing datastore;
converting the normalized second modality data into enhanced second modality data by:
converting the second delta table of the second schema into a second delta table of the third schema, and
storing the second delta table of the third schema in the third processing datastore;
associating the normalized first modality data with the normalized second modality data in the second processing datastore; and
processing a multimodal data operation on a combination of the enhanced first modality data and the enhanced second modality data.
